(19) United States
(12) Patent Application Publication
(10) Pub. No.: US 2025/0302658 A1
(43) Pub. Date: Oct. 2, 2025

(54) EXERCISE APPARATUS FOR PENIS

(71) Applicant: Hui LI, Songjiang District, Shanghai (CN)

(72) Inventor: Hui LI, Shanghai (CN)

(21) Appl. No.: 18/866,279

(22) PCT Filed: Apr. 26, 2023
(86) PCT No.: PCT/CN2023/090899

§ 371 Date: Nov. 15, 2024

(30) Foreign Application Priority Data
May 16, 2022 (CN) 202210528219.3

(51) Int. Cl. A61F 5/41; A61F 7/00; A61F 7/02

(52) U.S. Cl. CPC A61F 5/41; A61F 7/02; A61F 2005/412; A61F 2007/0048

(57) ABSTRACT

A penis exercise device for exercising penis, which comprises a heating device, a stretching device, and a vacuum device, can exercise the penis. By using this device for exercise, men can achieve the goal of increasing penis length and thickness. It can be used to solve the problem of short reproductive organs of the male.

### FIELD

The present invention relates to a device for penis exercising and enlarging.

### BACKGROUND

The invention involved in this patent does not violate social morality. China National Food and Drug Administration has issued a document to classify penile enlargement devices as Class III medical devices for management: https://www.nmpa.gov.cn/directory/web/nmpa/xxgk/fgwj/gzwj/gzwjylqx/20160701163101826.html

The penis is an important organ of the male urinary and reproductive system. A healthy penis can not only perform normal physiological functions, but also play an important role in enhancing male self-confidence. Research shows that the average length of a Chinese male penis is 8 cm when it is soft and 11-16 cm when it is erect. If it is less than 5 cm when it is soft and less than 8 cm when it is erect, it is considered a short penis. A short penis may cause disharmony in conjugal relations and exert great psychological pressure on men.

At present, methods to solve the problem of short penis include penis lengthening surgery, penis exercise device and other technologies. The effect of penis lengthening surgery is not ideal, and it is prone to damage the tissue structure of penis, which may cause sexual dysfunction in seriously cause loss in severe cases.

I have also applied for patents for some penis exercise device before, but there are many problems, the operation is complicated, and the growth rate is slow.

### SUMMARY

The purpose of the present invention is to provide exercise devices and associated devices for the penis, solve the problems of the existing device, provide better devices and methods for exercising penis than the existing penis exercisers, and achieve better penis enlargement effects, also solve the problem of short penis.

The present invention aims to achieve the purpose as follows:

A penis heating device, which is used for penis exercise, comprises at least a heating component and a control component, which are connected, said heating component can be tubular in shape, and both ends of the tube are penetrable, and the control component can control the power supply and heating of the heating component. The cross-section of said tubular component is not necessarily a standard circle and can be of other shapes. The axis of the tube may not necessarily be a straight line, it can also be a curve. Said term “penetrable” means that there are no obstructions at both ends of the tubular heating component when in use, and penises or penises wearing other devices (such as vacuum pump tubes) can pass through them. If the tubular heating component has a temporary cover, when the cover is removed, both ends are open, then it is also “penetrable”, and a penis can pass through it. The heating component is powered by electricity to generate heat.

Said control component can be a simple temperature control switch or a control circuit board with a temperature control switch or a temperature probe (temperature sensor).

The temperature probe can be non-contact or contact.

Preferably, the temperature probe adopts an infrared detection temperature probe.

Preferably, the temperature probe detects the temperature of the penis skin.

Optionally, the probe has an elastic element or has elasticity, which presses the probe onto the skin of the penis.

The temperature probe can also detect the temperature on the heating element, thus indirectly controlling the temperature of the penis.

Preferably, said heating component is made of a material that can release electromagnetic waves, and when powered on, it generates a heating effect by releasing electromagnetic waves. Preferably, said electromagnetic wave is far-infrared.

Optionally, the wall of said heating component has a cavity, which can contain fluid, and the inner side of the wall in contact with the penis is made of soft material.

Optionally, the tube wall is connected to a fluid charging device, which can charge fluid into the cavity to make the soft part of the inner wall expand towards the axis of the tube. So that the penis can be wrapped around.

Optionally, said heating component is a soft, rollable heating sheet or heating film.

Optionally, said heating component is a soft, rollable, and planar heating blanket. The heating blanket has a cavity inside, which can contain fluid. The heating element can be located inside the cavity.

Said heating sheet, heating film, and heating blanket can be rolled into a tube shape and placed around the penis.

Preferably, the number of heating components is greater than 1, and their thicknesses are different. The thicker ones are sleeved outside the thinner ones, and their relative positions can be adjusted relatively. Preferably, there are electrodes connected between each heating component.

Preferably, for adjacent heating components, one has a guide block and the other has a guide groove, and the guide block can move in the guide groove. Preferably, strip electrodes are placed in the guide groove, and the guide block has contact electrodes.

Optionally, the number of heating components is greater than 1, and they can be connected end-to-end in series. Preferably, adjacent heating components are provided with electrode plugs and electrode sockets that are mutually matching.

Optionally, for adjacent heating components, there are structures with fixed mutual positions between them, such as elastic clamping structures, threaded structures, and various mechanical clamping structures that exist in existing markets, textbooks, and publicly available technical materials.

A penis heating device, comprises at least a heating element, a fluid container, a control component, and a fluid charging device; said fluid container can be filled with fluid; said control component is connected to the heating element to control the heating element for fluid heating; said fluid container has flexible material, and it can be formed into a tube, the penis can be placed in the tube; the fluid charging device can be connected to the fluid container, when the fluid is filled into the fluid container, the fluid container expands inward and presses to the penis closely. Said fluid may be air, liquid or a combination thereof. Preferably, both ends of said tube are penetrable.

Preferably, the aforementioned device is used to heat the penis while the penis is under tension.

Preferably, the device is used to heat the penis while the penis is subject to expansion force.

Preferably, the device is used to heat the penis while the penis is under force.

Preferably, said component is provided with a temperature probe or a temperature control switch.

Preferably, the target temperature range for the penis to be heated by the device is between 40 and 47 degrees Celsius.

The control component can keep the temperature of the penis within the range of 40-47 degrees after heating. Preferably, the optimal range is 42-44 degrees.

Preferably, the heating component comprises at least two semi-tubular or partially tubular heating components to form a whole tube.

They can be hinged together and rotated to form a tube, or they can be formed by inserting a pin into a hole.

Said partial tubular shape refers to several parts obtained when a tube is divided into more than three parts.

A penis exercise device, provided with a device capable of stretching the penis (available on the market, textbooks and existing publicly available technical materials) and any one of the penis heating devices mentioned above.

A penis exercise device, provided with a device capable of expanding penis (available on the market, textbooks and existing publicly available technical materials) and any one of the penis heating devices mentioned above.

The specific structure of the penile stretching device mentioned above can refer to the existing penile stretching devices on the market and publicly available technical information on penile stretching devices. Most of them are characterized by a fixing component and a tension-providing component. The fixing component are usually fixed on the penis, mostly near the coronal sulcus of the glans penis. The tension-providing component are used to provide the force required to stretch the penis and are connected to the fixing component.

Generally, there are these types of fixing component: rotary fixing component (the included angle between different parts is reduced by the rotation of the rotating shaft, so as to clamp the penis inside), sliding or moving fixing component (the penis inside is clamped by the relative sliding or moving of different parts), belt clamping component (the belt is clamped and fixed around the penis), vacuum negative pressure fixing component (vacuum negative pressure is formed near the glans to fix the penis), and internal inflation airbag fixing (an annular air bag is placed around the penis, and the periphery of the air bag is fixed by hard material. After inflating, the airbag can only expand inward to clamp the penis).

Types of tension-providing components: hanging heavy objects (providing tension by gravity), retractable brackets that can be fixed relatively to the body to provide tension (stretching the penis by extending the bracket after fixation), component that generate tension by movement, vacuum negative pressure stretching component (stretching the penis by placing it in a vacuum negative pressure container), and combinations of the above component.

The above-mentioned penile expansion device can be: a vacuum negative pressure device (placing the penis in a vacuum negative pressure container to expand it), or a device that can clamp the root of the penis (after clamping, the front end of the penis can be expanded by applying a push force and a pull force in the direction of the glans penis).

A method for exercising penis is provided, which applies force to penis while heating the part of the penis subjected to the force.

A method for exercising penis is provided, which applies tension to penis while heating the part of the penis that is being pulled.

A method for exercising penis is provided, which applies vacuum suction to penis while heating it.

A method for exercising penis is provided, which heats penis while it is subjected to expansion force.

The expansion force can be generated when the penis blood is pushed to the front end of the penis by pushing force after clamping the root of the penis, or it can be generated when vacuum negative pressure is applied to the penis.

The above-mentioned heating component adopts an electric heating method, which can be an electric heating wire, electric heating tube, electrode type, or electric heating film type, and is provided with corresponding heating elements. It can be contact heating or non-contact heating (thermal radiation).

Heating wires and heating sheets can be pressed on plastic films to make heating films. When using, roll up the heating film into a tube shape and wrap it around the penis. Preferably, a cloth wrap can be provided outside the heating film to enhance comfort.

Preferably, a metal layer or metal material is arranged between the heating component and the testicles.

The fluid charging device can be an electric air pump, a hand-held soft inflatable ball (with a one-way valve), a manual piston pump, and any fluid charging device on the market, textbooks and existing publicly available technical materials. The fluid can be gas or liquid. Optionally, the fluid charging device is provided with a one-way valve.

Provides a seat, which has the common characteristics of a general chair or stool, and is characterized in that it is used for exercising the penis and has a bracket, and the rope for stretching the penis can move around the supporting point on the bracket.

Preferably, that position of the support point is provided with a pulley which is installed on the bracket, and the rope can bypass the pulley to drive the pulley to rotate.

Preferably, when in use, the position of the support point is higher than or equal to the horizontal position of the penis root of the user.

A support for a seat is provided, which is used for exercising penis. It has tubular or cylindrical rods that are interconnected to form a bracket. The rope used to stretch the penis can move around the support point on the bracket, and it has a fixing device that can be fixed to a chair or stool.

Preferably, that position of the support point is provided with a pulley which is installed on the bracket, and the rope can bypass the pulley to drive the pulley to rotate.

Preferably, when in use, the position of the support point is higher than or equal to the horizontal position of the penis root of the user.

Any of the above-mentioned seats and brackets is characterized in that it is provided with a rope for connecting heavy objects and the penis. The heavy object is used to generate tension that stretches the penis.

The structure of the above-mentioned fixing device can refer to the existing structure of fixing mechanical parts in the mechanical industry.

A penis exercise device is provided, which comprises at least a vacuum device and a container, said vacuum device is connected to the container and can pump out the fluid (gas or liquid) in the container to form vacuum negative pressure, and the penis can be placed inside the container, it is characterized in that it has a heating element that can heat the penis while the penis is subjected to vacuum negative pressure. Said heating device can be located inside or outside the container.

Optionally, said heating element is generally tubular in shape. Preferably, it has a control component for controlling the temperature of the penis after heating.

Preferably, any of the penile exercise devices is characterized in that the target temperature range of the penis after being heated is between 40 and 47 degrees Celsius.

The control component can keep the temperature of the penis within the range of 40-47 degrees Celsius after heating. Preferably, the optimal range is 42-44 degrees Celsius.

A penis exercise device is provided, which comprises at least a vacuum device and a container. The vacuum device is connected to the container and can pump out the fluid (gas or liquid) in the container to form vacuum negative pressure, and the penis can be placed inside the container. It is characterized in that the contact position between the container and the penis root is provided with an inward (that is, toward the penis axis) hard protrusion.

After the penis is engorged and swollen, the hard protrusions can exert a radial clamping force on the penis directed toward the inside of the penis.

The hard protrusion can be directly connected or integrated with the container, or assembled from several parts, or indirectly connected to the container through soft materials. To improve comfort and sealing, the inner side of the hard protrusion can be made of soft material.

A penis exercise device is provided, which comprises at least a vacuum device and a container. The vacuum device is connected to the container and can pump out the fluid (gas or liquid) in the container to form vacuum negative pressure, and the penis can be placed inside the container. It is characterized in that the position where the container contacts the base of the penis is provided with a soft sleeve, and the periphery of the soft sleeve is fixedly sealed and connected to the hard container, and the inside of the soft sleeve can surround the penis. On the outer periphery of the device surrounding the penis, there are tightenable fixing parts. After the fixing parts are tightened, the soft sleeve can be tightly pressed around the penis.

The tightenable fixing parts refers to a device that can reduce the circumference of the contact part with the soft sleeve.

The structure of the fixing device described here may refer to the fixing components provided with the aforementioned penis stretching device.

When in use, it is not necessary to lock the root first. After the penis is swollen to a certain extent by congestion, lock the root.

The aforementioned heating device can be integrated with the aforementioned penile exercise device (vacuum device), or made detachable and separable.

To improve the aforementioned penis exercise device with a vacuum device and the existing penis vacuum negative pressure exercise device of the prior art, it also has a negative pressure sensor, and the control component is connected to the negative pressure sensor. When the control component detects that the absolute value of negative pressure drops (or drops to a certain value), the vacuum device operates to restore the negative pressure value to the set value or nearby.

Preferably, the vacuum device adopts an electric vacuum pump.

Preferably, the target temperature range for heating the penis with the above heating devices is between 40 and 47 degrees Celsius.

Preferably, the control components of the above heating devices can control the target temperature to be near the constant temperature set by the user.

Any of the above devices that use vacuum negative pressure to exercise the penis is characterized in that it can use liquid, which is placed in a container (such as water), and it has heating components that can heat the liquid and heat the penis through the liquid.

A temperature control method for a penis heating device is provided, characterized in that it uses a temperature sensor to detect the temperature of the penis or heating element, and the user's target temperature is T1-T2, which includes at least the following steps:
a. At the beginning, power the heating element with the highest voltage Umax of the device, causing the heating element to quickly heat up and reach the target temperature T1-T2;
b. Supply power to the heating element for a certain period of time with the existing voltage Ux=(Umin+Umax)/2;
c. Detect the actual temperature Tx, and if it reaches the target temperature T1˜T2, return to step B; If Tx<T1, Umin=Ux, return to step B; If Tx>T2, Umax=Ux, return to step B.

A temperature control method for a penis heating device is provided, characterized in that it uses a temperature sensor to detect the temperature of the penis or heating element, and the user's target temperature is T1-T2, which includes the following steps:
A1. Power the heating element with the highest system voltage Ux=Umax, causing the heating element to rapidly heat up and reach the target temperature T1-T2;
B1. Heat for a certain time with voltage Ux=Ux−Uz (Uz is the voltage change);
C1. Detect the actual temperature Tx and determine that T1<Tx<T2;
D1. If unfounded, return to step B1;
E1. If founded, heat with voltage Ux for a certain period of time;
F1. Detect the actual temperature Tx and determine that T1<Tx<T2;
G1. If founded, return to step E1; If Tx<T1, heat with voltage Ux=Ux+Uz for a certain period of time; Return to step F1; If Tx>T2, return to step B1.

A temperature control method for a penis heating device is provided, characterized in that it uses a temperature sensor to detect the temperature of the penis or heating element, and the user's target temperature is T1-T2, which includes the following steps:
A2. Power the heating element with the highest system voltage Ux=Umax, causing the heating element to rapidly heat up and reach the target temperature T1-T2, Ux=Umin;
B2. Power the heating element with the existing voltage Ux=Ux+Uz, and heat for a certain period of time;
C2. Detect the actual temperature Tx and determine that T1<Tx<T2; If founded, proceed to step D2; If unfounded, return to step B2;
D2. Power the heating element with the existing voltage Ux and heat it for a certain period of time;
E2. Detect the actual temperature Tx and determine that T1<Tx<T2; If founded, return to step D2; If Tx<T1, return to step B2; If Tx>T2, heat for a certain period of time with the existing voltage Ux=Ux Uz, and return to step C2.

A penis heating device is provided, which comprises at least a control component and a heating components, and the control components adopts at least one of the aforementioned temperature control methods.

The above-mentioned vacuum devices can be a hand-held airbag (which may have one-way valves), a manual piston pump, an electric vacuum pump and other vacuum pump devices available on the market, teaching books and published technical materials.

The soft materials described in this paper can be soft materials such as soft rubber materials, silica gel, latex, TPE, TPR, etc., or other soft materials currently available on the market or in public technical documents.

The above devices with heating function can be provided with a heat insulation layer and radiation protection layer.

The insulation layer has a hole and is made of insulation material such as silicone, cotton products, ceramic fiber paper, etc. The radiation protection layers can be made of metal materials. These two functional layers can be integrated.

Heating elements and components can be any of the following: far-infrared heating, resistance heating, electric heating alloys, electric heating materials, microwave heating devices, electromagnetic wave heating, light heating, electromagnetic induction heating devices, electric heating wires, electric heating plates, electric heating belts, electric heating cables, electric heating plates, electric couples, electric heating coils, electric heating rods, electric heating belts, electric heating cores, mica heating plates, ceramic heating plates, tungsten molybdenum products, silicon carbon rods, molybdenum powder, tungsten bars, electric heating wires, mesh belts, and other materials that are currently available on the market and publicly available technical data.

All the devices mentioned above can be provided with fixing straps for hanging or fixing the device on the body, such as the waist, neck, shoulders and other parts.

Preferably, all the devices mentioned above are provided with LCD screens, which can display the values of pressure or intensity of pressure, temperature and tension.

Preferably, the above-mentioned devices are provided with wireless communication devices for wireless connection and communication with mobile phones, which can display pressure or numerical values of pressure, temperature, and tension on the mobile phone. Preferably, the wireless communication device is a Bluetooth device.

### Advantages

Using the device and method of the present invention to exercise the penis is safe and reliable, and can avoid many shortcomings of the prior art, such as foreskin edema, foreskin congestion, pee-hole bleeding, penile skin folliculitis, cysts, foreskin elongation and other problems, and the penis grows faster and has better growth effect.

### BRIEF DESCRIPTION OF THE DRAWINGS

The present invention will be further explained in conjunction with the accompanying drawings and embodiments.

FIG. 1 3D view of the assembly in Embodiment 1.

FIG. 2 Sectional view of the assembly in Embodiment 2.

FIG. 3 3D view of the assembly in Embodiment 2.

FIG. 4 Exploded view of the assembly in Embodiment 2.

FIG. 5 3D view of the assembly in Embodiment 3.

FIG. 6 Exploded view of the assembly in Embodiment 3.

FIG. 7 3D view of the snap spring 12 in Embodiment 3.

FIG. 8 3D view of the clamp 11 in Embodiment 3.

FIG. 9 Sectional view of the clamp 11 in Embodiment 3.

FIG. 10 3D view of the clamp 9 in Embodiment 3.

FIG. 11 Exploded view of the sealing ring 9 in Embodiment 4.

FIG. 12 3D view of the vacuum tube 13 in the assembly in Embodiment 4.

FIG. 13 Sectional view of the vacuum tube 13 in Embodiment 4.

FIG. 14 3D view of the assembly in Embodiment 5.

FIG. 15 3D view of the assembly in Embodiment 6.

FIG. 16 Sectional view of the assembly in Embodiment 6.

FIG. 17 3D view of the inner container 34 in Embodiment 6.

FIG. 18 3D view of the assembly in Embodiment 7.

FIG. 19 Sectional view of the assembly in Embodiment 7.

FIG. 20 3D and sectional view of the assembly in Embodiment 8.

FIG. 21 3D view of the inner heating tube in Embodiment 8.

FIG. 22 3D view of the inner and outer heating tubes in Embodiment 8.

FIG. 23 3D view of Embodiment 9.

FIG. 24 3D view of the backup heating tubes 59 and 60 in Embodiment 9.

FIG. 25 3D view of Embodiment 10.

FIG. 26 3D view of Embodiment 11.

FIG. 27 Expanded view of the heating film 52 used in the outer heating tube in Embodiment 8.

FIG. 28 Program logic diagram of Embodiment 12.

FIG. 29 Program logic diagram of Embodiment 13.

FIG. 30 Program logic diagram of Embodiment 14.

FIG. 31 3D view of the assembly in Embodiment 4.

FIG. 32 Physical diagram of Embodiment 15.

FIG. 33 Physical diagram of Embodiment 16.

FIG. 34 Physical diagram of Embodiment 17.

FIG. 35 Physical diagram of Embodiment 18.

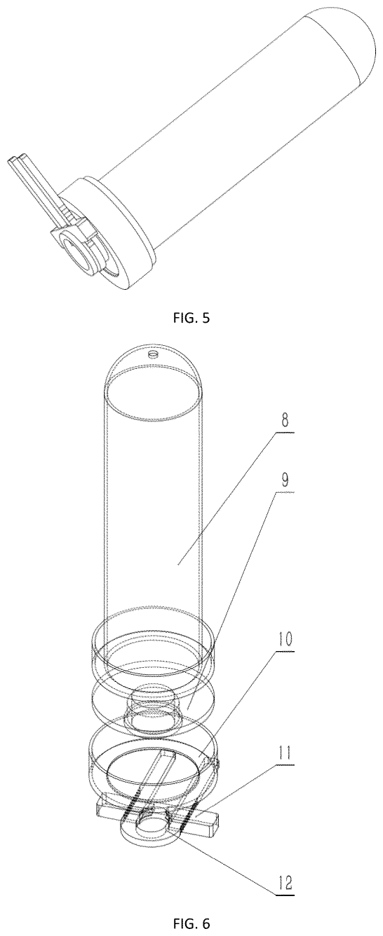

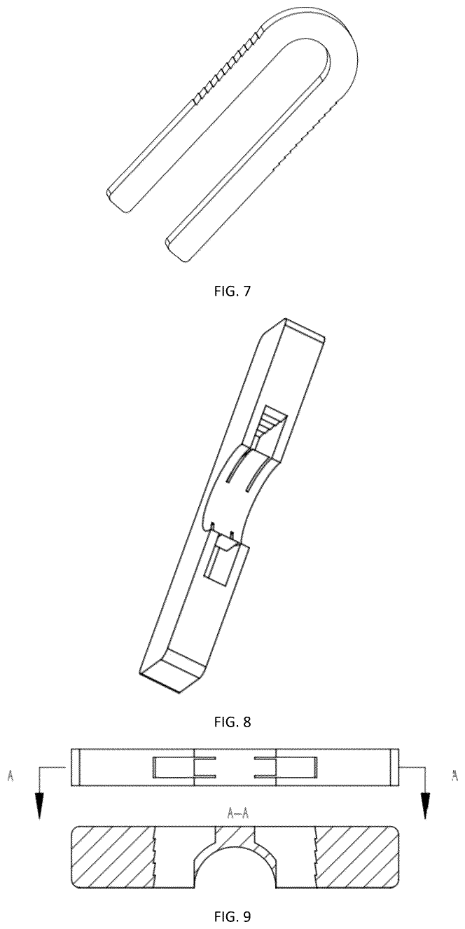

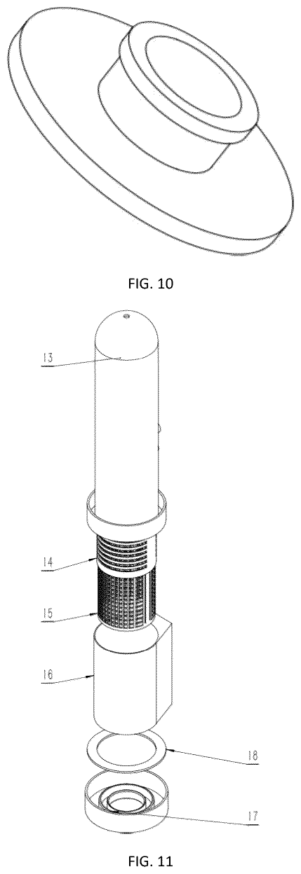

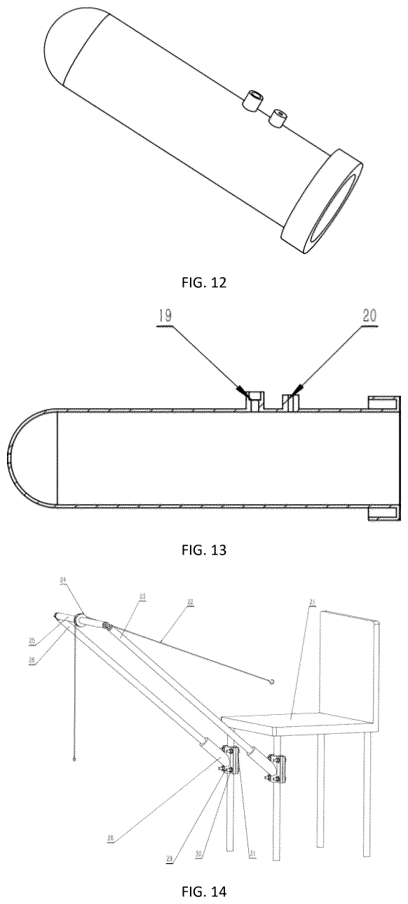

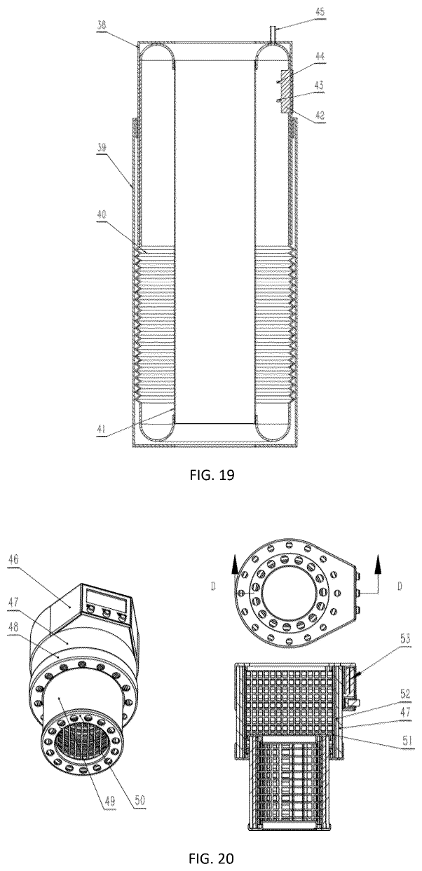

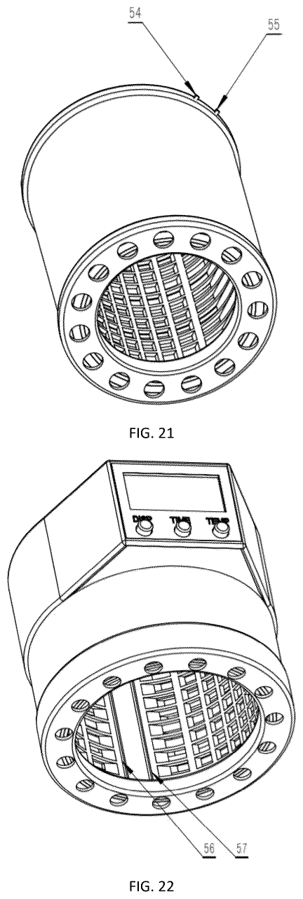

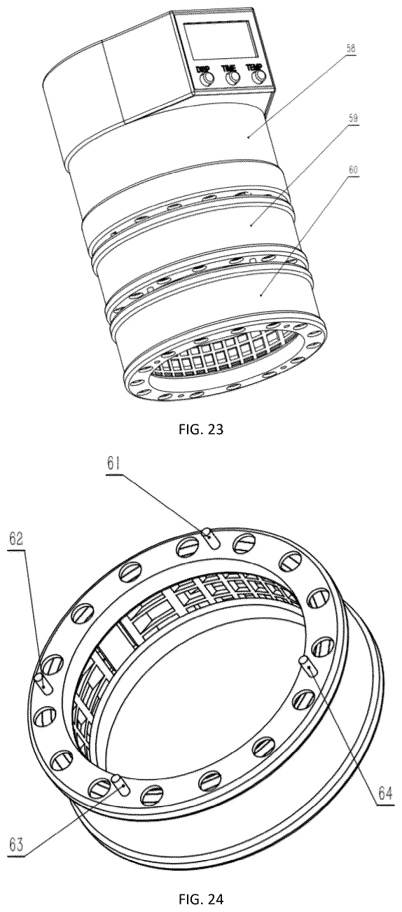

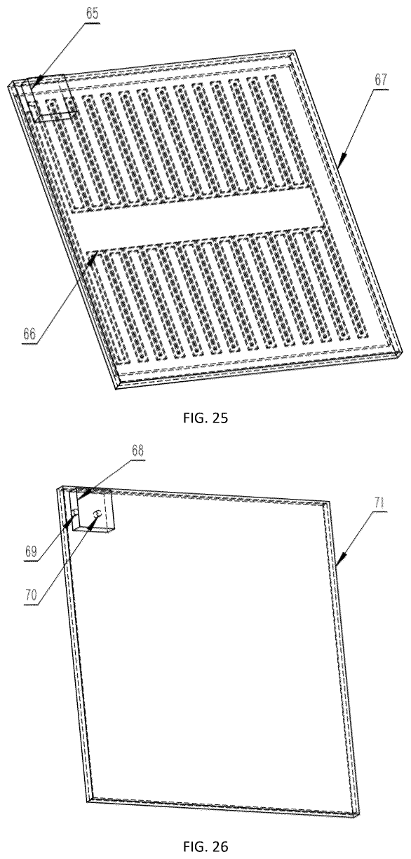

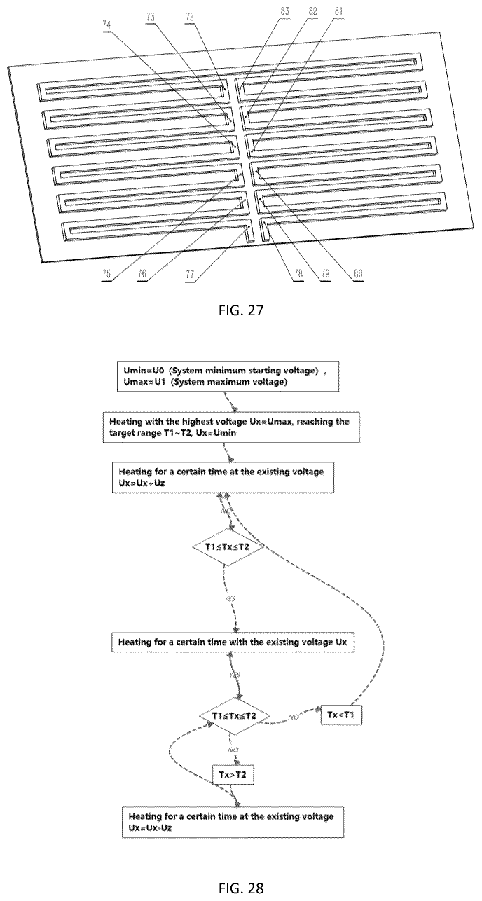

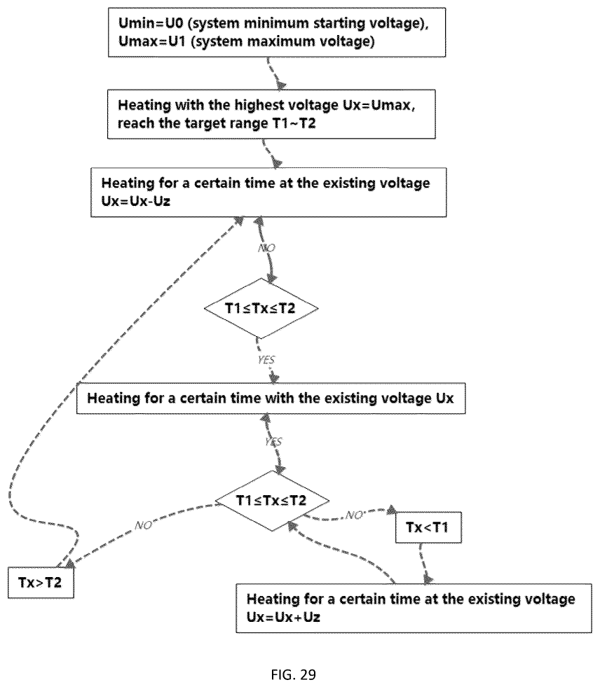

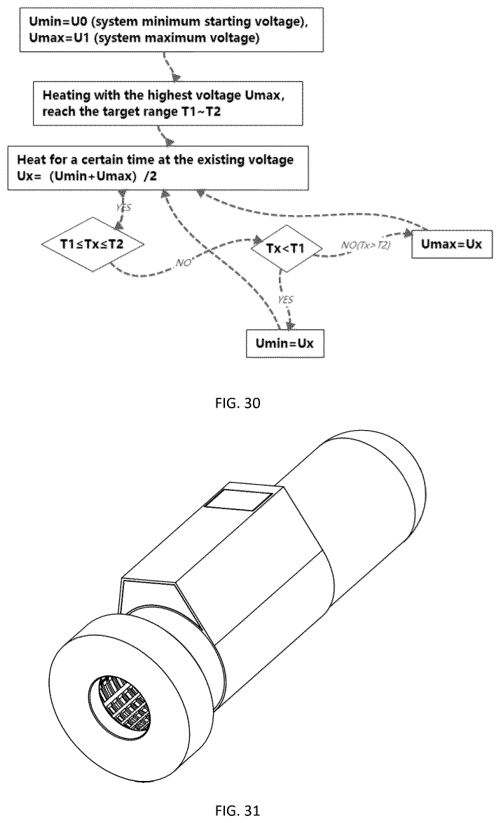

In FIG. 2: 1—Pumping ball; 2—Hose; 3—Vacuum tube; 4—End cap.
In FIG. 4: 5—Vacuum tube; 6—Sealing ring; 7—End cap;
In FIG. 6: 8—Vacuum tube; 9—Sealing ring; 10—End cap; 11—Clamp; 12—Snap spring;
In FIG. 11: 13—Vacuum tube; 14—Heating film; 15—Fixing mesh; 16—Control component; 17—End cap; 18—Sealing ring;
In FIG. 13: 19—Pressure sensor socket; 20. Temperature sensor socket;
In FIG. 14: 21—Chair; 22—Traction rope; 23—Strut; 24—Pulley; 25—strut; 26—Strut; 28—Fixed cover; 29—Bolt; 30. Nut; 31—Fixed cover;
In FIG. 16: 32—Pipe joint; 33—Shell; 34—Inner tank; 35—Electrode; 36—Electrode; 37—Control component;
In FIG. 19: 38—Inner shell; 39—Outer shell; 40. Inner container; 41—Flexible film; 42—Control component; 43—44—Electrodes; 45—Pipe joints;
In FIG. 20: 46—Control component; 47—Outer shell; 48—End cover; 49—Inner shell; 50. End cover; 51—Fixing mesh; 52—Heating film; 53—Circuit board;
In FIG. 21: 54—electrode; 55—Electrode;
In FIG. 22: 56—57—Guide groove;
In FIG. 23: 58—Main heater; 59—60. Backup heating tube;
In FIG. 24: 61—Fixed pin; 62—Electrode plug; 63—Fixed pin; 64—Electrode plug;
In FIG. 25: 65—Control component; 66—Heating film; 67—Clothjacket;
In FIG. 26: 68—Control component; 69.70—Electrodes; 71—Jacket;
In FIG. 27: 72˜83 Electrode contacts.
In FIGS. 28-29: U0—The lowest starting voltage of the system; U1—The highest voltage of the system; Ux—Real-time heating voltage; T1—The lower limit of the target temperature; T2—the upper limit of the target temperature; Uz—Step voltage (voltage value with each change); Tx—Real-time measured temperature value.
In FIG. 30: Umin, voltage real-time lower limit; Umax, voltage real-time upper limit; others as above.

### EMBODIMENTS

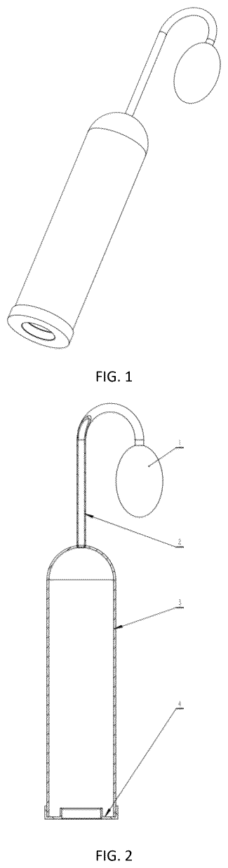

Embodiment 1 is an embodiment of the present invention, which exercises the penis by applying vacuum negative pressure to the penis. In the embodiment shown in FIGS. 1 and 2: the pumping ball 1 (vacuum device) is connected to the vacuum tube 3 (container) through the hose 2, with an end cap 4 at the lower end of the vacuum tube 3, which are sealed and connected (which can be connected and sealed by glue). The inner diameter of the end cap 4 is smaller than that of the vacuum tube 3. The vacuum tube 3 and the end cap 4 are made of hard plastic. The pumping ball 1 is made of rubber and has a one-way valve for exhaust. The fluid here is air. During use, put the penis into the vacuum tube 3, and press the end cap 4 against the skin at the root of the penis (to improve sealing, soft rubber can be added here). Repeatedly squeezing the pumping ball 1 can form vacuum negative pressure in the vacuum tube 3, causing the penis to become congested and expand. When the outer diameter of the penis base is greater than the inner diameter of the end cap 4, the penis root will be clamped by the end cap 4, and the blood will no longer flow out easily. End caps or products are available in multiple sizes (inner diameters) to accommodate different penis thicknesses.

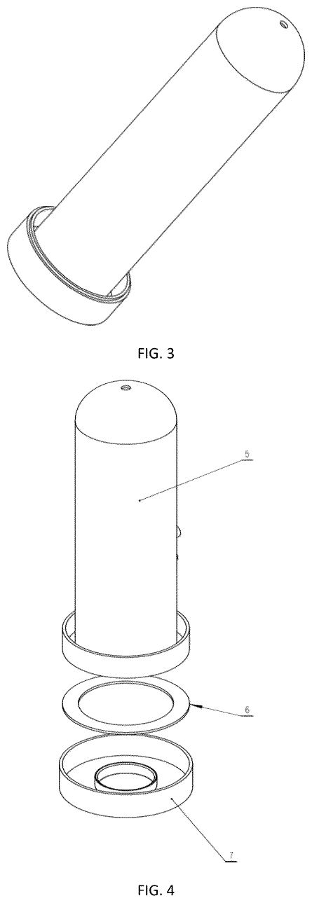

Embodiment 2 is another embodiment of the vacuum negative pressure penis exercise device of the present invention. In the embodiment shown in FIGS. 3 and 4: both the vacuum tube 5 and the end cap 7 are made of hard plastic with matching threads, and there is a sealing ring 6 between them. When they are tightened, they can be sealed. In this case, simply provide end caps with different inner diameters to accommodate different penis sizes.

In the above two embodiments, the end caps 4 and 7 are hard protrusions.

Embodiment 3 is another embodiment of the vacuum negative pressure penis exercise device of the present invention. In the embodiment shown in FIGS. 5˜10 and Embodiment 2, the difference is that the end cap 10 has a larger inner diameter and the seal ring 9 is shaped differently with a short tubular portion that can fit over the root of the penis. The two legs of the snap spring 12 are elastic and can be clamped on the ratchet teeth of the clamp 11 after rebounding so that they are relatively fixed. When they are clamped on the short tube of the sealing ring 9, the blood at the front end of the penis can be prevented from flowing back into the body. When in use, first use the snap spring 12 and the clamp 11 to slightly clamp the root of the penis, exhaust the air in the vacuum tube 8 through the vacuum device, so that the penis is engorged and swollen, and then use the snap spring 12 and the clamp 11 to tightly clamp the penis root. After exercising, press the two legs of snap spring 12 inward to release it.

Embodiment 4 is another embodiment of the vacuum negative pressure penis exercise device of the present invention. In Embodiment 4 shown in FIGS. 11˜13: a heating film 14 is provided in the vacuum tube 13, and inside the heating film 14 is a fixing mesh 15 for fixing the shape of the heating film 14. The control component 16 is installed outside the vacuum tube 13 and includes circuit board, temperature sensor, air pressure sensor, LCD screen, buttons, power socket and other components, used to control the heating temperature of the penis and the vacuum negative pressure in the vacuum tube 13. The sealing ring 18 is located between the bottom of the vacuum tube 13 and the end cap 17 (with a hard protrusion). The end cap 17 is screwed onto the threads at the bottom of the vacuum tube 13. The pressure sensor is sealed and installed in the pressure sensor jack 19 for detecting the negative pressure value in the vacuum tube 13, and the infrared temperature sensor is installed in the temperature sensor jack 20, and they are both connected to the control component 16. Preferably, the vacuum device adopts an electric vacuum pump installed at the top of vacuum tube 13, which is connected to control component 16. The heating film 14 is also connected to the control component 16. The control component 16 can control the pressure inside the vacuum tube and the heating temperature of the penis based on the values measured by temperature and pressure sensors.

Embodiment 5 is an embodiment of the seat and bracket of the present invention. In FIG. 14, the struts 23, 25, and 26 are fixed by bolts and nuts, forming a bracket. The strut 26 is installed inside the tube on fixed cover 28, and so is the strut 23. The fixed cover 28 and the fixed cover 31 are fixed to the legs of the chair 21 through four bolts 29 and nuts 30. The pulley 24 is mounted on the strut 25. The traction rope 22 goes around the pulley 24, the right end of which is connected to the penis stretching device, and the left end is connected to a heavy object used to provide tension. The penis stretching device is connected to the user's penis, and the user sits on a chair. The support point in the figure is the midpoint of the strut 25. The fixed cover 28, 31, bolt 29, and nut 30 are the fixing parts.

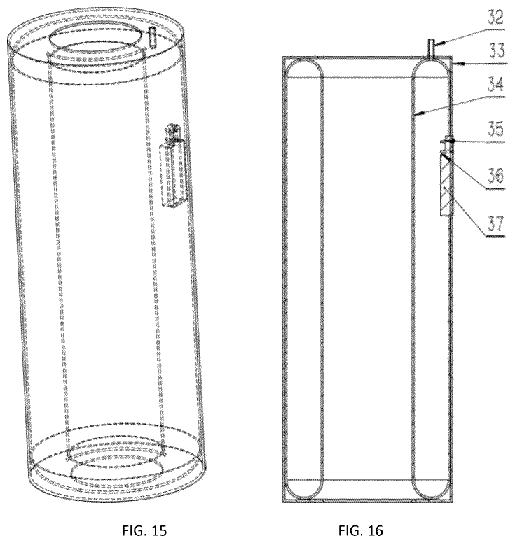

Embodiment 6 is an embodiment of the penis-heating device of the present invention. In Embodiment 6 shown in FIGS. 15˜17, the inner container 34 is made of soft material (such as latex, silicone, etc.) and is filled with fluid. The inner container 34 is installed in the hard shell 33, and its two ends and periphery are limited by the shell 33. The inner container 34 is provided with a pipe joint 32, which is connected to a fluid charging device (such as an air pump, an inflatable ball, a hydraulic pump, etc.). After filled with fluid, the inner container 34 will expand inward (the axis of the shell 33), to wrap around the penis placed inside. The control component 37 is connected to the heating electrodes 35 and 36 to control the heating of the liquid inside the inner container 34, thereby controlling the heating temperature of the penis. Here, the inner container 34 and the heating electrodes 35 and 36 constitute a heating component. The penis, the traction rope for stretching the penis and the stretcher fittings can all pass through the through hole in the inner container 34.

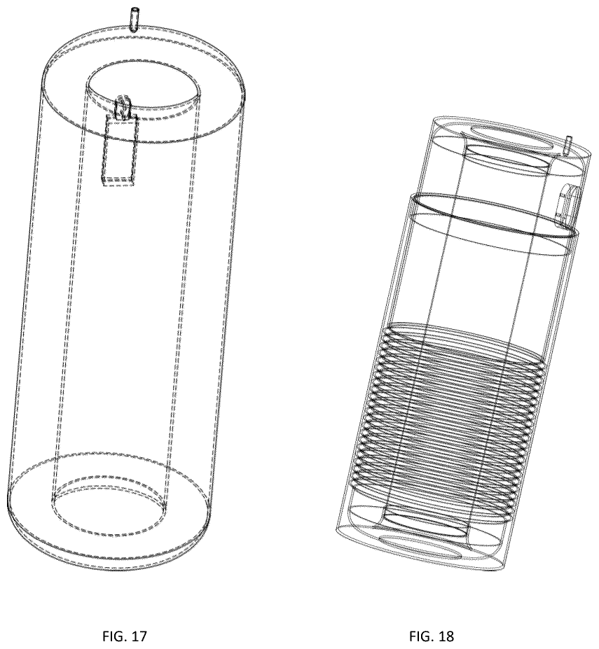

Embodiment 7 is an embodiment of the penis-heating device of the present invention. In Embodiment 7 shown in FIGS. 18˜19, the inner shell 38 can move within the outer shell 39, and they can be fixed by friction, or by other positioning structures (such as threads, positioning pins, existing mechanical industry positionable structures, etc.). The inner container 40 is placed in the limiting structure composed of the inner shell 38 and the outer shell 39, so that it cannot expand around, but can only expand inward. The inner container 40 is made of slightly hard but somewhat elastic-plastic, and its lower part is a corrugated structure, so it can expand or compress in the axial direction. The flexible film 41 is made of soft and elastic materials (such as latex, rubber, etc.). The inner container 40 and the flexible film 41 are fixedly bonded together by glue to form a closed cavity, where fluid can enter and exit through the pipe joint 45. The control component 42 can control the heating of the fluid in the cavity by the electrodes 43 and 44. When in use, the user first adjusts the relative position of the inner shell 38 and the outer shell 39 according to the length of his penis, and the fluid charging device (not shown here for the convenience of drawing) injects fluid into the cavity through the pipe joint 45. When the fluid is injected, the inner container 40 expands axially until it contacts with the top of the outer shell 39 and the inner shell 38, and the flexible film 41 contacts with the penis, so that the penis can be heated after the fluid is heated.

Embodiment 8 is an embodiment of the penis heating device of the present invention, which consists of inner and outer heating tubes with different inner diameters. In Embodiment 8 shown in FIGS. 20, 21, and 22, the inner shell 49 can move within the outer shell 47 to accommodate penises of different lengths. The control component 46 is installed on the outer shell 47 and connected to the heating film 52 to control the temperature of the heating film 52, to control the temperature of the penis placed inside. Heating film 52 can release far-infrared radiation when powered on, heating the penis placed inside. The heating film 52 is installed inside the outer shell 47, and the fixing mesh 51 is placed inside the heating film 52 to fix the shape of the heating film 52. In the inner heating tube shown in FIG. 21, two electrodes are connected to the heating film in the inner heating tube; in the outer heating tube shown in FIG. 22, there are two guide grooves 56 and 57 in the fixing mesh. The electrodes 54, and 55 cooperate with the guide grooves 56, and 57 and can slide in the guide grooves. The electrodes 54, and 55 function as electrode contacts and also as guide blocks. Inside the guide grooves 56 and 57 are the strip-shaped electrodes 72-83 of the heating film shown in FIG. 27. When the electrodes 54 and 55 come into contact with the strip-shaped electrodes at different positions, the heating film in the corresponding areas will be heated.

Embodiment 9 is an embodiment of the penis heating device of the present invention, and its internal structure is similar to that of Embodiment 8, except that several heating pipes are connected in series instead of being nested inside and outside as in Embodiment 8. In Embodiment 9 shown in FIGS. 23˜24, the main heater 58 has control components and can be operated and used independently. 59 and 60 are backup heating tubes that can be connected in series with the main heater to accommodate penises of different lengths. The spare heating pipes are provided with electrode plugs 62 and 64, fixing pins 61 and 63, and matching electrode jacks and positioning jacks. There are matching electrode sockets and positioning sockets at the bottom of the main heater. One end of the electrode plug is connected to the heating film, and the other end is connected to the electrode jack at the higher level. The electrode jack can be directly connected with the electric heating film at this level or connected with the electrode plug at this level.

Embodiment 10 is an embodiment of the penis-heating device of the present invention. In Embodiment 10 shown in FIG. 25, the control component 65 is connected to the heating film 66 to control its heating. The cloth jacket 67 is placed outside the heating film 66. The heating film and the cloth jacket are all made of soft materials, which can be bent into a tubular shape and wrapped around the penis to heat the penis (the outside can be enclosed and fixed with Velcro). In this way, a contact temperature sensor can be used to make contact with the penis, thereby controlling the heating temperature of the penis.

Embodiment 11 is an embodiment of the penis-heating device of the present invention. In Embodiment 11 shown in FIG. 26, the outer sleeve 71 is made of soft material, and a sealed cavity is formed inside, and the cavity is filled with liquid. The control component 68 is installed on the jacket 71 to control the heating of the liquid by the electrodes 69 and 70, thereby controlling the temperature of the penis. When in use, it can be bent into a tubular shape and wrapped around the penis to heat the penis (the outside can be enclosed and fixed with Velcro).

Embodiments 12, 13, and 14 shown in FIGS. 28, 29, and 30 are logic diagrams of the temperature control program used in the penis heating device. They mainly provide three methods of how to achieve approximately constant temperature.

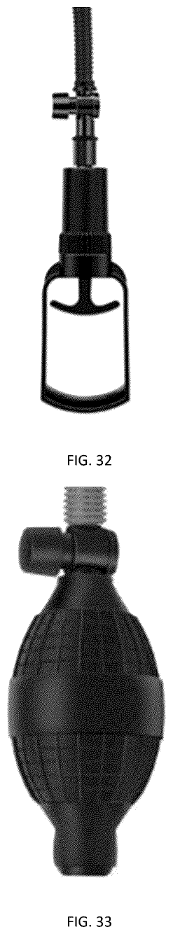

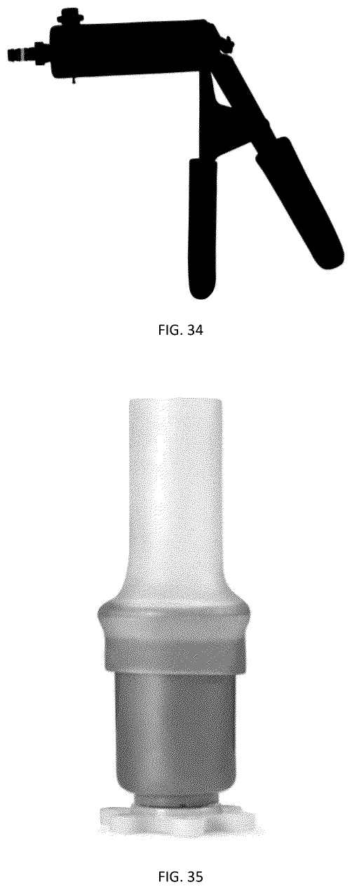

FIGS. 32-34 are physical pictures of three types of vacuum devices. FIGS. 32 and 34 adopt a piston pump structure to achieve vacuum pumping. The pumping ball shown in FIG. 33 is made of elastic rubber balls.

FIG. 35 shows a fixing device for a penis stretching device, with the upper part made of elastic soft rubber material, which is wrapped around the penis and pressed tightly to it. The lower part is victimized by a piston pump. After the bottom is vacuumed, the upper part press against the penis closely, and can be pulled down to exert a downward pulling force on the penis.

For the convenience of drawing, the drawings of connecting pipes, vacuum pumping devices, control components and other parts are omitted or simplified, but it does not mean that these parts do not exist.

For the convenience of drawing, common supporting components that can be completed by ordinary industry personnel, such as air valves (manual or electric), inflation devices, vacuum negative pressure pumps, inflation pumps, control components, control circuits, wires, power supplies (commonly used power sources such as batteries, dry batteries, direct insertion power supplies, etc.), sensors, etc., are omitted here, but it does not mean that they do not exit.

The terms of parts, assemblies, components, members, etc., as described herein, have no special limitations or particular definitions or meanings concerning the number of parts or relationships contained herein, and are not limited to the concepts of mechanical textbooks. The concepts of equipment, instruments, devices, etc. described in this paper have no special requirements on the number or complexity of their components and are not limited by textbook concepts.

1. A penis heating device, which can be used for penis exercise, and which comprises at least a heating component and a control component, which are connected, said heating component can be tubular in shape, and both ends of the tube are penetrable, and the control component can control the power supply and heating of the heating component, the term “penetrable” means that there are no obstructions at both ends of the tubular heating component when in use, and penises or penises wearing other devices can pass through them.

2. The penis heating device, as described in claim 1, wherein the wall of the heating component has a cavity, which can contain fluid, and the inner side of the wall in contact with the penis has of soft material.

3. The penile heating device, as described in claim 1, wherein the heating component is a soft, rollable planar heating sheet, heating film, or heating blanket.

4. The penis heating device, as described in claim 1, wherein the number of heating components is greater than 1, and their thicknesses are different; the thicker ones are sleeved outside the thinner ones, and their relative positions can be adjusted relatively.

5. The penis heating device, as described in claim 1, wherein the number of heating components is greater than 1, and they can be connected end-to-end in series.

6. A penis heating device, which comprises at least a heating element, a fluid container, a control component, and a fluid pressurizing device; said fluid container can be filled with fluid; said control component is connected to the heating element to control the heating element for fluid heating; said fluid container has flexible material, and it can be formed into a tube, and the penis can be placed in the tube; said fluid pressurizing device can be connected with the fluid container, so that the fluid can be filled into the fluid container, so that the fluid container expands inward and presses to the penis closely.

7. The penis heating device, as described in claim 6, both ends of the tube are penetrable.

8. The penis heating device, as described in claim 1, wherein the penis is heated using the device while the penis is under tension.

9. The penis heating device, as described in claim 1, wherein the penis is heated using the device while being subjected to an expansion force.

10. The penis heating device, as described in claim 1, wherein the target temperature range for penis heating is between 40 and 47 degrees Celsius.

11. The penis heating device, as described in claim 1, wherein the heating component comprises at least two semi-tubular or partially tubular heating components to form a whole tube.

12. A penis exercise device, provided with a device capable of stretching the penis, which also has any penis heating device, as described in claim 1.

13. A penis exercise device, provided with a device capable of expanding penis, which also has any penis heating device, as described in claim 1.

14. A penis exercise device, which comprises at least a vacuum device and a container, said vacuum device is connected to the container and can pump out the fluid in the container to form a vacuum negative pressure, and the penis can be placed inside the container, wherein it has a heating element that can heat the penis while the penis is subjected to vacuum negative pressure.

15. A penis exercise device, as described in claim 14, wherein the heating element is tubular in shape.

16. A penis exercise device, as described in claim 14, which is provided with a control device for controlling the temperature of the penis after heating, and the target temperature range for penis heating is between 40 and 47 degrees Celsius.

17. A penis exercise device, which comprises at least a vacuum device and a container, the vacuum device is connected to the container and can pump out the fluid in the container to form vacuum negative pressure, and the penis can be placed inside the container, wherein the contact position between the container and the penis root is provided with an inward (that is, toward the penis axis) hard protrusion.

18. A penis exercise device, which comprises at least a vacuum device and a container, the vacuum device is connected to the container and can pump out the fluid in the container to form vacuum negative pressure, and the penis can be placed inside the container, wherein the position where the container contacts the base of the penis is provided with a soft sleeve, and the periphery of the soft sleeve is fixedly sealed and connected to the hard container, and the inside of the soft sleeve can surround the penis, on the outer periphery of the device surrounding the penis, there are tightenable fixing parts, after the fixing parts are tightened, the soft sleeve can be tightly pressed around the penis.

19. The penis heating device, as described in claim 14, which is provided with a negative pressure sensor, and the control device is connected to the negative pressure sensor; when the control device detects that the absolute value of the negative pressure drops (or drops to a certain value), the vacuum device operates to restore the negative pressure value to the set value.

20. The penis heating device, as described in claim 2, wherein the number of heating components is greater than 1, and their thicknesses are different; the thicker ones are sleeved outside the thinner ones, and their relative positions can be adjusted relatively.
